(19) United States
(12) Patent Application Publication
(10) Pub. No.: US 2022/0244246 A1
(43) Pub. Date: Aug. 4, 2022

(54) INTESTINAL BIOMARKERS FOR GUT HEALTH IN DOMESTICATED BIRDS

(71) Applicant: DUPONT NUTRITION BIOSCIENCES APS, COPENHAGEN K (DK)

(72) Inventors: Alexander BEKELE-YITBAREK, Wilmington, DE (US); Marion BERNARDEAU, Caen (FR); Venessa EECKHAUT, MERELBEKE (BE); Kirsty GIBBS, Cheshire (GB); Flip VAN IMMERSEEL, MERELBEKE (BE)

(21) Appl. No.: 17/600,339

(22) PCT Filed: Mar. 31, 2020
(86) PCT No.: PCT/US2020/025922

§ 371 Date: Sep. 30, 2021

Related U.S. Application Data
(60) Provisional application No. 62/827,606, filed on Apr. 1, 2019.

(51) Int. Cl. G01N 33/50; C12Q 1/04; C12Q 1/6883; C12Q 1/689; G01N 33/53

(52) U.S. Cl. CPC G01N 33/5091; C12Q 1/04; G01N 33/5308; C12Q 1/689; C12Q 1/6883

(57) ABSTRACT

Provided herein, inter alia, are methods for measuring and assessing intestinal health in poultry. The disclosed metabolic biomarkers and associated methods for identifying and quantifying the same are reliable, rapid and, in some embodiments, non-invasive, and can be used to provide information with respect to the gut health of poultry, such as chickens.

### CROSS-REFERENCE OF RELATED APPLICATIONS

The present application is a U.S. National Stage Application under 35 U.S.C. § 371 of International Application No. PCT/US2020/025922, filed Mar. 31, 2020, which claims priority to U.S. Provisional Patent Application No. 62/827,606, filed on Apr. 1, 2019, and are incorporated by reference herein in their entireties.

### FIELD OF THE INVENTION

Provided herein, inter alia, are methods for measuring and assessing intestinal health in domesticated birds.

### BACKGROUND

In poultry species, the gastrointestinal tract not only is involved in digestion and absorption, but also interacts with the immune system to promote good health. The lumen of the intestinal tract is coated with a thin layer of sticky, viscous mucous, and embedded in this mucus layer, are millions and millions of bacteria and other microbes. When the intestinal bacteria are in balance (i.e., the good bacteria outnumber the bad bacteria), the gut is said to be healthy. A healthy microbiota provides the host with multiple benefits, including colonization resistance to a broad spectrum of pathogens, essential nutrient biosynthesis and absorption, and immune stimulation that maintains a healthy gut epithelium and an appropriately controlled systemic immunity. In settings of “dysbiosis” or disrupted symbiosis, microbiota functions can be lost or deranged, resulting in increased susceptibility to pathogens, altered metabolic profiles, or induction of proinflammatory signals that can result in local or systemic inflammation or autoimmunity. Thus, the intestinal microbiota of poultry plays a significant role in the pathogenesis of many diseases and disorders, including a variety of pathogenic infections of the gut such as coccidiosis or necrotic enteritis.

Quantifiable and easy-to-measure biomarkers for diagnosing or predicting the intestinal health of poultry do not yet exist but would be of tremendous value as a tool to monitor and/or prognose clinical and subclinical intestinal entities that cause or are correlated with performance problems in flocks and to evaluate control methods for intestinal health, independent of whether the triggers are derived from host, nutritional, or microbial factors. The subject matter disclosed herein addresses these needs and provides additional benefits as well.

### SUMMARY

Provided herein, inter alia, are methods for measuring and assessing intestinal health in poultry. The disclosed metabolic biomarkers and associated methods for identifying and quantifying the same are reliable, rapid and, in some embodiments, non-invasive, and can provide information with respect to the gut health of poultry, such as chickens.

Accordingly, in some aspects, provided herein are methods for determining the intestinal health status of a domesticated bird comprising: detecting and/or quantifying one or more (such as any of 1, 2, 3, 4, 5, 6, 7, 8, 9, 10, 11, 12, 13, 14, 15, 16, 17, 18, 19, or 20) metabolite(s) in a fecal and/or intestinal content sample from the bird selected from the group consisting of linoleyl carnitine, linalool, 3-[(9Z)-9-octadecenoyloxy]-4-(trimethylammonio)butanoate, (-)-trans-methyl dihydrojasmonate, icomucret, 1,3-dioctanoylglycerol, ethyl 2-nonynoate, L-arginine, 4-aminobutyrate, 2-amino-isobutyrate, D-alpha-aminobutyrate, cadaverine, putrescine, uracil, hypoxanthine, D-alanine, sarcosine, methional, hexanal, and malondialdehyde, wherein an increased level of said one or more metabolite(s) in said fecal or intestinal content sample, when compared to the level found in fecal or intestinal content samples of healthy control animals, is an indicator of poor intestinal health. In some embodiments, the method further comprises detecting and/or quantifying one or more (such as any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 ,37, 38, 39, 40, 41, 42, 43, 44, or 45) metabolite(s) in a fecal and/or intestinal content sample from the bird selected from the group consisting of 5-(2-carboxyethyl)-2-hydroxyphenyl beta-D-glucopyranosiduronic acid, 4,15-Diacetoxy-3-hydroxy-12,13-epoxytrichothec-9-en-8-yl 3-hydroxy-3-methylbutanoate, scoparone, asp-leu, ethyl benzoylacetate, L-(+)-glutamine, 1-allyl-2,3,4,5-tetramethoxybenzene, (DL)-3-O-methyldopa, dictyoquinazol A, 1-(3-furyl)-7-hydroxy-4,8-dimethyl-1,6-nonanedione, methyl 3,4,5-trimethoxycinnamate, butylparaben, aspartic acid, glutamic acid, L-pyroglutamic acid, L-glutamine, L-histidine, glycine, (−)-beta-pineen, L-asparagine, L-homoserine, L-serine, L-threonine, L-proline, L-tyrosine, L-leucine, dopamine, taurocholic acid, typtamine, tauroursodeoxychdic acid, glycoursodeoxycholic acid, ursodeoxycholic acid, cholic acid, nonanal, 3-methyl-2-butenal, DL-glyceraldehyde, allantoin, nicotinic acid, N-acetylglucosamine, spermidine, (dimethlyamino)acetonitrile, glycoursodeoxycholic acid, tauroursodeoxycholic acid, cortisol, and heptanal, wherein a decreased level of said one or more metabolite(s) in said fecal or intestinal content sample, when compared to the level found in fecal or intestinal content samples of healthy control animals, is an indicator of poor intestinal health. In some embodiments of any embodiment described herein, the intestinal content sample is derived from colon. In some embodiments, the method further comprises detecting and/or quantifying L-alanine, wherein a decreased level of L-alanine in said colon content sample, when compared to the level found in colon content samples of healthy control animals, is an indicator of poor intestinal health. In some embodiments of any embodiment described herein, the method further comprises detecting and/or quantifying acetylcarnitine, wherein an increased level of acetylcarnitine in said colon content sample, when compared to the level found in colon content samples of healthy control animals, is an indicator of poor intestinal health. In some embodiments of any embodiment described herein, the intestinal content sample is derived from caecum. In some embodiments, the method further comprises detecting and/or quantifying L-alanine, wherein an increased level of L-alanine in said caecum content sample, when compared to the level found in caecum content samples of healthy control animals, is an indicator of poor intestinal health. In some embodiments of any embodiment described herein, the method further comprises detecting and/or quantifying acetylcarnitine, wherein a decreased level of acetylcarnitine in said caecum content sample, when compared to the level found in caecum content samples of healthy control animals, is an indicator of poor intestinal health. In some embodiments of any embodiment described herein, the method further comprises detecting and/or quantifying populations of one or more microorganism(s) in a fecal and/or intestinal content sample from the bird selected from the group consisting of: a microorganism from the Clostridiales vadinBB60 group family of microorganisms and a microorganism from the Peptostreptococcaceae family of microorganisms, wherein a decreased population of said one or more microorganism(s) in said fecal or intestinal content sample, when compared to the level found in fecal or intestinal content samples of healthy control animals, is an indicator of poor intestinal health. In some embodiments, the method further comprises detecting and/or quantifying populations of one or more (such as any of 1, 2, 3, 4, 5, 6, 7, 8, 9, or 10) microorganism(s) in a fecal and/or intestinal content sample from the bird selected from the group consisting of: a microorganism from the genus Brevibacterium, Brachybacterium, Ruminiclostridium, Candidatus Arthromitus, Ruminococcus with the exception of Ruminococcus torques, Streptococcus, Shuttleworthia, Lachnospiraceae NK4A136 group, and Ruminococcaceae UCG-005, wherein a decreased population of said one or more microorganism(s) in said fecal or intestinal content sample, when compared to the level found in fecal or intestinal content samples of healthy control animals, is an indicator of poor intestinal health. In some embodiments of any embodiment described herein, the intestinal content sample is obtained from ileum, colon, or caecum. In some embodiments of any embodiment described herein, the method further comprises detecting and/or quantifying populations of one or more (such as any of 1, 2, or 3) microorganism(s) in an intestinal content sample from the bird selected from: a microorganism from the genus Defluviitaleaceae UCG-011, a microorganism from the genus Lachnoclostridium, or a microorganism from the Ruminococcus torques group, (a) wherein a decreased population of said one or more microorganism(s) obtained from the caecum, when compared to the level found in caecum samples of healthy control animals, is an indicator of poor intestinal health; and/or (b) wherein an increased population of said one or more microorganism(s) obtained from the colon, when compared to the level found in colon samples of healthy control animals, is an indicator of poor intestinal health. In some embodiments of any embodiment described herein, the method further comprises detecting and/or quantifying populations of one or more microorganism(s) in an intestinal content sample from the bird a microorganism from the genus Lactobacillus, (a) wherein an increased population of said one or more microorganism(s) obtained from the caecum, when compared to the level found in caecum samples of healthy control animals, is an indicator of poor intestinal health; and/or (b) wherein a decreased population of said one or more microorganism(s) obtained from the colon, when compared to the level found in colon samples of healthy control animals, is an indicator of poor intestinal health. In some embodiments of any embodiment described herein, intestinal health is determined by one or more of (a) measuring villus length in the duodenum of the birds; (b) measuring villus-to crypt ratio in the duodenum of the birds; (c) measuring T-lymphocyte infiltration in villi; and/or (d) scoring the macroscopic gut appearance of the birds. In some embodiments of any embodiment described herein, the domesticated bird is selected from the group consisting of chickens, turkeys, ducks, geese, quail, and pheasant. In some embodiments, the chicken is a broiler. In some embodiments of any embodiment described herein, said one or more metabolite(s) are quantified by using antibodies which specifically bind to said metabolite. In some embodiments, said antibodies are part of an Enzyme-Linked Immuno Sorbent Assay (ELISA). In some embodiments of any embodiment described herein, said one or more metabolite(s) are quantified by using gas chromatography-mass spectrometry (GC-MS), nuclear magnetic resonance (NMR), liquid chromatography-mass spectrometry (LC-MS) or HPLC.

In another aspect, provided herein is a method for detecting and/or quantifying one or more metabolite(s) from a domesticated bird at risk for or thought to be at risk for poor intestinal health comprising: detecting and/or quantifying one or more (such as any of 1, 2, 3, 4, 5, 6, 7, 8, 9, 10, 11, 12, 13, 14, 15, 16, 17, 18, 19, 20, 21, or 22) metabolites in a sample selected from the group consisting of linoleyl carnitine, linalool, 3-[(9Z)-9-octadecenoyloxy]-4-(trimethylammonio)butanoate, (−)-trans-methyl dihydrojasmonate, icomucret, 1,3-dioctanoylglycerol, ethyl 2-nonynoate, L-arginine, 4-aminobutyrate, 2-amino-isobutyrate, D-alpha-aminobutyrate, cadaverine, putrescine, uracil, hypoxanthine, D-alanine, sarcosine, methional, hexanal, malondialdehyde, L-alanine, and acetylcarnitine, wherein the sample is a fecal or an intestinal content sample. In some embodiments, the method further comprises detecting and/or quantifying one or more (such as any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 ,37, 38, 39, 40, 41, 42, 43, 44, or 45) metabolites in the sample selected from the group consisting of 5-(2-carboxyethyl)-2-hydroxyphenyl beta-D-glucopyranosiduronic acid, 4,15-Diacetoxy-3-hydroxy-12,13-epoxytrichothec-9-en-8-yl 3-hydroxy-3-methylbutanoate, scoparone, asp-leu, ethyl benzoylacetate, L-(+)-glutamine, 1-allyl-2,3,4,5-tetramethoxybenzene, (DL)-3-O-methyldopa, dictyoquinazol A, 1-(3-furyl)-7-hydroxy-4,8-dimethyl-1,6-nonanedione, methyl 3,4,5-trimethoxycinnamate, butylparaben, aspartic acid, glutamic acid, L-pyroglutamic acid, L-glutamine, L-histidine, glycine, (−)-beta-pineen, L-asparagine, L-homoserine, L-serine, L-threonine, L-proline, L-tyrosine, L-leucine, dopamine, taurocholic acid, typtamine, tauroursodeoxychdic acid, glycoursodeoxycholic acid, ursodeoxycholic acid, cholic acid, nonanal, 3-methyl-2-butenal, DL-glyceraldehyde, allantoin, nicotinic acid, N-acetylglucosamine, spermidine, (dimethlyamino)acetonitrile, glycoursodeoxycholic acid, tauroursodeoxycholic acid, cortisol, and heptanal. In some embodiments of any of the embodiments disclosed herein, the method further comprises detecting and/or quantifying populations of one or more microorganism(s) in a fecal and/or intestinal content sample from the bird selected from the group consisting of: a microorganism from the Clostridiales vadinBB60 group family of microorganisms and a microorganism from the Peptostreptococcaceae family of microorganisms. In some embodiments of any of the embodiments disclosed herein, the method further comprises detecting and/or quantifying populations of one or more (such as any of 1, 2, 3, 4, 5, 6, 7, 8, 9, or 10) microorganism(s) in a fecal and/or intestinal content sample from the bird selected from the group consisting of: a microorganism from the genus Brevibacterium, Brachybacterium, Ruminiclostridium, Candidatus Arthromitus, Ruminococcus with the exception of Ruminococcus torques, Streptococcus, Shuttleworthia, Lachnospiraceae NK4A136 group, and Ruminococcaceae UCG-005. In some embodiments of any of the embodiments disclosed herein, the method further comprises detecting and/or quantifying populations of one or more (such as any of 1, 2, or 3) microorganism(s) in an intestinal content sample from the bird selected from: a microorganism from the genus Defluviitaleaceae UCG-011, a microorganism from the genus Lachnoclostridium, or a microorganism from the Ruminococcus torques group. In some embodiments of any of the embodiments disclosed herein, the method further comprises detecting and/or quantifying populations of one or more microorganism(s) in an intestinal content sample from the bird a microorganism from the genus Lactobacillus. In some embodiments of any of the embodiments disclosed herein, the intestinal content sample is obtained from ileum, colon, or caecum. In some embodiments of any of the embodiments disclosed herein, the method further comprises (a) measuring villus length in the duodenum of the birds; (b) measuring villus-to crypt ratio in the duodenum of the birds; (c) measuring T-lymphocyte infiltration in villi; and/or (d) scoring the macroscopic gut appearance of the birds. In some embodiments of any of the embodiments disclosed herein, the domesticated bird is selected from the group consisting of chickens, turkeys, ducks, geese, quail, emus, ostriches, and pheasant. In some embodiments, the chicken is a broiler. In some embodiments of any of the embodiments disclosed herein, said one or more metabolite(s) and/or said populations of one or more microorganism(s) are quantified by using antibodies which specifically bind to said metabolite. In some embodiments, said antibodies are part of an Enzyme-Linked Immuno Sorbent Assay (ELISA). In some embodiments of any of the embodiments disclosed herein, said one or more metabolite(s) are quantified by using gas chromatography-mass spectrometry (GC-MS), nuclear magnetic resonance (NMR), liquid chromatography-mass spectrometry (LC-MS) or HPLC. In some embodiments of any of the embodiments disclosed herein, said one or more microorganisms are identified and quantified by real-time PCR. In some embodiments, the method further comprises sequencing the 16S ribosomal DNA (rDNA) gene.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIG. 1A is a bar graph depicting body weight (g) in control (ctrl.) and challenged chickens at day 26. FIG. 1B is a bar graph depicting coccidiosis and dysbiosis scores in control (ctrl.) and challenged chickens at day 26.

FIG. 2A is a plot depicting intestinal villus height (μm) in control (CTRL) compared to challenged chickens. FIG. 2B is a plot depicting crypt depth (μm) in control (CTRL) compared to challenged chickens. FIG. 2C is a plot depicting the ratio of villus height/crypt depth in control (CTRL) compared to challenged chickens.

FIG. 3A is a graph depicting the association between intestinal villus length (pm) and body weight (g) in challenged (dark) and control (light) birds. FIG. 3B is a graph depicting the association between intestinal crypt depth (pm) and body weight (g) in challenged (dark) and control (light) birds. FIG. 3C is a graph depicting the association between the ratio of villus height/crypt depth and body weight (g) in challenged (dark) and control (light) birds.

FIG. 4A is a plot depicting the area percentage of immune cell (CD3+) infiltration of intestinal tissue in control (CTRL) compared to challenged chickens. FIG. 4B is a graph depicting the association between the area percentage of immune cell (CD3, area %) infiltration of intestinal tissue with body weight (g) in challenged (dark) and control (light) birds. FIG. 4C is a graph depicting the association between the area percentage of immune cell (CD3, area %) infiltration of intestinal tissue with coccidiosis score in challenged (dark) and control (light) birds. FIG. 4D is a graph depicting the association between the area percentage of immune cell (CD3, area %) infiltration of intestinal tissue with dysbiosis score in challenged (dark) and control (light) birds. FIG. 4E is a graph depicting the association between the area percentage of immune cell (CD3, area %) infiltration of intestinal tissue with villus length (pm) in challenged (dark) and control (light) birds.

FIG. 5A is a bar graph depicting body weight (g) in control (ctrl.) and challenged chickens at day 28. FIG. 5B is a bar graph depicting coccidiosis and dysbiosis scores in control (ctrl.) and challenged chickens at day 28.

FIG. 6A and FIG. 6B are bar graphs depicting the identity and quantity of non-limiting examples of metabolites measured in the colon (FIG. 6A) and caecum (FIG. 6B) of challenged and control birds.

FIG. 7A and FIG. 7B are bar graphs depicting the identity and quantity of non-limiting examples of metabolites measured in the colon (FIG. 7A) and caecum (FIG. 7B) of challenged and control birds.

### DETAILED DESCRIPTION

For domesticated birds, particularly for birds bred for food production, a well-functioning intestinal tract is of key importance for digestion and nutrient absorption and consequently low feed conversion, and is also crucial for health and welfare. Indeed, intestinal diseases and syndromes are common in some commercial forms of poultry, such as broilers, and constitute the most important cause for treatment (Casewell et al., 2003). In poultry farming, coccidiosis is by far the most important intestinal disease (Yegani and Korver, 2008; Caly et al., 2015). Clinical diseases caused by bacterial pathogens are not common, but it is widely recognized that a variety of intestinal syndromes can affect broiler performance, including subclinical necrotic enteritis and coccidiosis, viral enteritis, and various non-defined enteritis syndromes (Yegani and Korver, 2008). It is not evident how to diagnose these subclinical entities and differentiate these from performance problems that have no infectious etiology, such as those caused by suboptimal formulated diets that not always cause intestinal damage.

The invention disclosed herein is based, inter alia, on the inventors' observations that the identity and quantity of constituent metabolites in the gut (i.e., intestines) and feces of poultry varies in accordance with intestinal health status. As such, identifying and quantifying metabolic species present in the chicken gut and/or fecal material can be used to monitor and/or prognose clinical and subclinical intestinal entities that cause or are correlated with performance problems (such as, but not limited to, decreased weight, poor feed conversion ratio (FCR), mortality, and altered intestinal structure and morphology).

### I. Definitions

As used herein, “microorganism” refers to a bacterium, a fungus, a virus, a protozoan, and other microbes or microscopic organisms.

The term “one or more metabolite(s)” as used herein refers to a single metabolite or to a plurality of metabolites, i.e. preferably at least 2, 3, 4, 5, 10, or 50 metabolites. It is to be understood that “metabolite” as used herein may be at least one molecule of said metabolite up to a plurality of molecules of the metabolite and that a plurality of metabolites means a plurality of chemically different molecules wherein for each metabolite at least one molecule up to a plurality of molecules may be present. A metabolite in accordance with the present invention encompasses all classes of organic or inorganic chemical compounds including those being comprised by biological material such as organisms (for example, microorganisms) or those produced as a consequence of the metabolism of an organism (for example, the metabolism of one or more microorganisms). In some embodiments, the metabolite in accordance with the present invention is a small molecule compound. In other embodiments, in case a plurality of metabolites is envisaged, said plurality of metabolites representing a metabolome, i.e. the collection of metabolites being comprised by an organism, an organ (such as the intestines), a tissue (such as intestinal tissue such as, but not limited to, colon or caecum tissue) or a cell at a specific time and under specific conditions.

The phrase “increased population of a metabolite when compared to the level found in samples from healthy control animals” means at least a 10-200% increase, such as any of about a 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or 200% increase, inclusive of all values falling in between these percentages. In some embodiments, the metabolite is not detectable at all in healthy control animals.

The phrase “decreased population of a metabolite when compared to the level found in samples from healthy control animals” means at least a 10-100% decrease, such as any of about a 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, 95%, or 100%, decrease, inclusive of all values falling in between these percentages. In some embodiments, the metabolite is not detectable at all in animals suffering from or thought to be suffering from poor intestinal health.

The term “poultry,” as used herein, means domesticated birds kept by humans for their eggs, their meat or their feathers. These birds are most typically members of the superorder Galloanserae, especially the order Galliformes which includes, without limitation, chickens, quails, ducks, geese, emus, ostriches, pheasant, and turkeys.

The term “intestinal health status” refers to the status of the gut wall structure and morphology which can be affected by, for example, infectious agents or a non-infectious cause, such as a suboptimal formulated diet. “Gut wall structure and morphology” can refer to, without limitation, epithelial damage and epithelial permeability which is characterized by a shortening of villi, a lengthening of crypts and an infiltration of inflammatory cells (such as, without limitation, CD3+ cells). The latter damage and inflammation markers can also be associated with a “severe” macroscopic appearance of the gut-compared to a “normal” appearance- when evaluated using a scoring system such as the one described by Teirlynck et al. (2011).

The phrase “poor intestinal health” refers to gut wall structure and morphology resulting from, for example, infectious agents or a non-infectious cause, such as a suboptimal formulated diet. A domesticated bird with poor intestinal health exhibits abnormal gut wall structure and morphology which is evidenced by, without limitation, one or more of epithelial damage and epithelial permeability characterized by one or more of shortening of villi, lengthening of crypts, and/or and an infiltration of inflammatory cells (such as, without limitation, CD3+ cells). The latter damage and inflammation markers can also be associated with a “severe” macroscopic appearance of the gut-compared to a “normal” appearance-when evaluated using a scoring system such as the one described by Teirlynck et al. (2011).

The term “fecal sample” refers to fecal droppings from birds.

The term “intestinal content sample” can refer to intestinal content obtained from, for example, necropsy of birds. The term “intestinal content at necropsy of birds” refers to a sample taken from the content present in one or more of the gizzard, ileum, caecum or colon, such as after said bird is euthanized. In other embodiments, “intestinal content sample” can refer to the contents of the intestines as well as the intestinal tissue itself. In further embodiments, “intestinal content sample” can refer to a sample obtained via mucosal scratching.

The phrase “quantifying populations of one or more metabolite(s) a fecal or intestinal content sample” refers to any method known to a person having ordinary skill in the art to quantify and/or identify said one or more metabolite(s) in the sample. Non-limiting examples of such methods include mass-spectrometrical methods, ELISA and mass spectrometry, or HPLC. It should be clear that the quantification of a single metabolite might be sufficient to determine intestinal health status but that also a combination of any of about 2, 3, 4, 5, 6, 7, 8, 9 or more metabolites can be used to determine the intestinal health status of the poultry.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of -10% to +10% of the numerical value, unless the term is otherwise specifically defined in context.

As used herein, the singular terms “a,” “an,” and “the” include the plural reference unless the context clearly indicates otherwise.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is also noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is further noted that the term “comprising,” as used herein, means including, but not limited to, the component(s) after the term “comprising.” The component(s) after the term “comprising” are required or mandatory, but the composition comprising the component(s) can further include other non-mandatory or optional component(s).

It is also noted that the term “consisting of,” as used herein, means including, and limited to, the component(s) after the term “consisting of.” The component(s) after the term “consisting of” are therefore required or mandatory, and no other component(s) are present in the composition.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

Other definitions of terms may appear throughout the specification.

### II. Methods

Provided herein are methods for determining the intestinal health status of a domesticated bird by detec ting and/or quantifying populations of one or more metabolite(s) in a fecal and/or intestinal content sample from a bird suffering from or thought to be suffering from poor intestinal health. As described in the Examples section, when poultry were administered therapeutic levels of antibiotics to induce dysbiosis followed by a cocktail containing opportunistic bacterial pathogens as well as a coccidial cocktail, statistically significant modulations in the quantity of these compounds occurred in the intestines of these chickens in comparison to the level of these compounds in untreated healthy controls. A variety of compounds were found to be differentially present in the colon and/or caecum of chickens challenged with dysbiosis versus the intestines of healthy untreated control animals. The types of compounds identified include, without limitation amino acids, bile salts, aldehydes, amines and other nitrogen-containing compounds, and alkenes.

In one non-limiting embodiment, the metabolite(s) are linoleyl carnitine ((3R)-3-[(9Z,12Z)-octadeca-9,12-dienoyl]oxy-4-(trimethylazaniumyl)butanoate), linalool (3,7-Dimethyl-1,6-octadien-3-ol), 3-[(9Z)-9-octadecenoyloxy]-4-(trimethylammonio)butanoate (O-oleoylcarnitine), (−)-trans-methyl dihydrojasmonate (Methyl [(1R,2R)-3-oxo-2-pentylcyclopentyl]acetate), icomucret ((5Z,8Z,11Z,13E,15S)-15-hydroxyicosa-5,8,11,13-tetraenoic acid), 1,3-dioctanoylglycerol ((2-hydroxy-3-octanoyloxypropyl) octanoate), ethyl 2-nonynoate (ethyl oct-2-ynoate), L-arginine, 4-aminobutyrate (gamma-Aminobutanoate), 2-amino-isobutyrate (2-azaniumyl-2-methylpropanoate), D-alpha-aminobutyrate, cadaverine (Pentane-1,5-diamine), putrescine (Butane-1,4-diamine), uracil, hypoxanthine (1H-purin-6(9H)-one), D-alanine, sarcosine (2-(Methylamino)acetic acid), methional (3-Methylsulfanylpropanal), hexanal (also called hexanaldehyde or caproaldehyde), and/or malondialdehyde (propanedial). When poultry were challenged with dysbiosis, a statistically significant increase in the quantity of these compounds occurred in the intestines of these chickens in comparison to the level of these compounds in untreated healthy controls. In some instances, the amount of the metabolite can exhibit at least a 10-200% increase in comparison to the level of this compound found in the intestines in untreated healthy controls, such as any of about a 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or 200% increase, inclusive of all values falling in between these percentages. In some embodiments, the metabolite is not detectable at all in healthy control animals. Any method known in the art can be used to quantify and identify the metabolites, such as, without limitation, antibody based assays (for example, ELISA or Western Blot), HPLC, or mass spec.

In alternative embodiments, the method can further include detecting and/or quantifying one or more of the following metabolites in a fecal and/or intestinal content sample from a bird suffering from or thought to be suffering from poor intestinal health: 5-(2-carboxyethyl)-2-hydroxyphenyl beta-D-glucopyranosiduronic acid (also known as, Dihydro Caffeic Acid 3-O-β-D-Glucuronide, a glucuronide metabolite of Caffeic acid), 4,15-Diacetoxy-3-hydroxy-12,13-epoxytrichothec-9-en-8-yl3-hydroxy-3-methylbutanoate (Mycotoxin T-2), scoparone (6,7-Dimethoxy-2H-chromen-2-one), asp-leu, ethyl benzoylacetate (ethyl 3-oxo-3-phenylpropanoate), L-(+)-glutamine, 1-allyl-2,3,4,5-tetramethoxybenzene (6-Methoxyelemicin), (DL)-3-O-methyldopa (2-Amino-3-(4-hydroxy-3-methoxyphenyl)propanoic acid), dictyoquinazol A (3-[2-(hydroxymethyl)-4-methoxyphenyl]-6-methoxyquinazolin-4-one), 1-(3-furyl)-7-hydroxy-4,8-dimethyl-1,6-nonanedione, methyl 3,4,5-trimethoxycinnamate (methyl (E)-3-(3,4,5-trimethoxyphenyl)prop-2-enoate), butylparaben (Butyl 4-hydroxybenzoate), aspartic acid, glutamic acid, L-pyroglutamic acid, L-glutamine, L-histidine, glycine, (−)-beta-pineen (6,6-Dimethyl-2-methylidenebicyclo[3.1.1]heptane), L-asparagine, L-homoserine, L-serine, L-threonine, L-proline, L-tyrosine, L-leucine, dopamine, taurocholic acid (2-{[(3α,5β,7α,12α)-3,7,12-trihydroxy-24-oxocholan-24-yl]amino}ethanesulfonic acid), typtamine (2-(1H-Indol-3-yl)ethanamine), tauroursodeoxychdic acid (2-[[(4R)-4-[(3R,5S,7S,8R,9S,10S,13R,14S,17R)-3,7dihydroxy-10, 13-dimethyl-2,3,4,5,6,7,8,9,11,12,14,15,16,17-tetradecahydro-1H- cyclopenta[a]phenanthren-17-yl]pentanoyl]amino]ethanesulfonic acid), glycoursodeoxycholic acid (2-[[(4R)-4-[(3R,5S,7S,8R,9S,10S,13R,14S,17R)-3,7-dihydroxy-10,13-dimethyl-2,3,4,5,6,7,8,9,11,12,14,15,16,17-tetradecahydro-1H-cyclopenta[a]phenanthren-17-yl]pentanoyl]amino]acetic acid), ursodeoxycholic acid (ursodiol; (4R)-4-[(3R,5S,7S,8R,9S,10S,13R,14S,17R)-3,7-dihydroxy-10,13-dimethyl-2,3,4,5,6,7,8,9,11,12,14,15,16,17- tetradecahydro-1H-cyclopenta[a]phenanthren-17-yl]pentanoic acid), cholic acid ((R)-4-((3R,5S,7R,8R,9S,10S,12S,13R,14S,17R)-3,7,12-Trihydroxy-10,13-dimethylhexadecahydro-1H-cyclopenta[a]phenanthren-17-yl)pentanoic acid), nonanal (also called nonanaldehyde, pelargonaldehyde or Aldehyde C-9), 3-methyl-2-butenal (3-methylbut-2-enal or 3-Methylcrotonaldehyde), DL-glyceraldehyde, allantoin ((2,5-Dioxo-4-imidazolidinyl) urea), nicotinic acid (niacin or Pyridine-3-carboxylic acid), N-acetylglucosamine, spermidine, (dimethlyamino)acetonitrile, tauroursodeoxycholic acid (2-[[(4R)-4-[(3R,5S,7S,8R,9S,10S,13R,14S,17R)-3,7-dihydroxy-10, 13-dimethyl-2,3,4,5,6,7,8,9,11,12,14,15,16,17-tetradecahydro-1H-cyclopenta[a]phenanthren-17-yl]pentanoyl]amino] ethanesulfonic acid), cortisol, and heptanal (heptanaldehyde). When poultry were challenged with dysbiosis, a statistically significant decrease in the quantity of these compounds occurred in the intestines of these chickens in comparison to the level of these compounds in untreated healthy controls. In some instances, the amount of the metabolite can exhibit at least a 10-100% decrease, such as any of about a 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, 95%, or 100%, decrease, inclusive of all values falling in between these percentages. In some embodiments, the metabolite is not detectable at all in animals suffering from or thought to be suffering from poor intestinal health. Any method known in the art can be used to quantify and identify the metabolites, such as, without limitation, antibody based assays (for example, ELISA or Western Blot), HPLC, or mass spec.

When using an intestinal content sample from a bird suffering from or thought to be suffering from poor intestinal health to detect and/or quantify one or more metabolites, the content sample can come from the colon. In further embodiments of the methods disclosed herein, the method can also include detecting and/or quantifying L-alanine in the colon. In this embodiment, a decreased level of L-alanine in the colon content sample, when compared to the level found in colon content samples of healthy control animals, is an indicator of poor intestinal health. In yet a further embodiment, the method can also include detecting and/or quantifying acetylcarnitine ((3R)-3-Acetoxy-4-(trimethylammonio)butanoate) in the colon. In this embodiment, an increased level of acetylcarnitine in the colon content sample, when compared to the level found in colon content samples of healthy control animals, is an indicator of poor intestinal health.

Moreover, when using an intestinal content sample from a bird suffering from or thought to be suffering from poor intestinal health to detect and/or quantify one or more metabolites, the content sample can come from the caecum. In further embodiments of the methods disclosed herein, the method can also include detecting and/or quantifying L-alanine in the caecum. In this embodiment, an increased level of L-alanine in the caecum content sample, when compared to the level found in caecum content samples of healthy control animals, is an indicator of poor intestinal health. In another embodiment, the method can also include detecting and/or quantifying acetylcarnitine ((3R)-3-Acetoxy-4-(trimethylammonio)butanoate) in the caecum. In this embodiment, a decreased level of acetylcarnitine in the caecum content sample, when compared to the level found in colon content samples of healthy control animals, is an indicator of poor intestinal health.

Also provided herein are methods for determining the intestinal health status of a domesticated bird by further quantifying populations of one or more microorganism(s) in a fecal and/or intestinal content sample from the bird. In one non-limiting embodiment, the microorganism(s) are selected from the Clostridiales vadinBB60 group family of microorganisms and/or a microorganism from the Peptostreptococcaceae family (e.g., Peptoclostridium difficile) of microorganisms.

Both the vadinBB60 group family and the Peptostreptococcaceae families of microorganisms are in the Clostridiales order of microorganisms and constitute a highly polyphyletic class of the phylum Firmicutes. Microbes in these families are gram positive and distinguished from the Bacilli by lacking aerobic respiration. Specifically, they are obligate anaerobes and oxygen is toxic to them (Bergey's manual of systematics of archaea and bacteria, Witman, Sup. Ed., Hoboken, N.J.: Wiley (2015); Galperin et al., 2016, Int. J. System. & Evol. Microbiol., 66:5506-13).

As described in the Examples section, when poultry were administered therapeutic levels of antibiotics to induce dysbiosis followed by a cocktail containing opportunistic bacterial pathogens as well as a coccidial cocktail, a statistically significant decrease in the population of vadinBB60 group family and Peptostreptococcaceae family microorganisms was observed in comparison to the level of these microorganisms that were found in samples obtained from healthy control animals.

Moreover, additional microorganisms were identified from the genera Brevibacterium, Brachybacterium, Ruminiclostridium, Candidatus Arthromitus, Ruminococcus (with the exception of Ruminococcus torques; for example, R. lactaiformans), Streptococcus, Shuttleworthia, Lachnospiraceae NK4A136 group, and Ruminococcaceae UCG-005. These microorganisms were also observed to significantly decrease in challenged birds in comparison to non-challenged control animals.

In additional embodiments, the method can also include identifying (i.e. detecting) and quantifying one or more microorganism from an intestinal content sample from the genus Defluviitaleaceae UCG-011, a microorganism from the genus Lachnoclostridium, or a microorganism from the Ruminococcus torques group. In this embodiment, a decreased population of one or more microorganism(s) of these genera in a sample obtained from the caecum is an indicator of poor intestinal health, when compared to the level found in caecum samples of non-challenged healthy control animals. However, an increased population of one or more microorganism(s) of these genera in a sample obtained from the colon is an indicator of poor intestinal health, when compared to the level found in colon samples of non-challenged healthy control animals.

In yet further embodiments, the method can also include identifying (i.e. detecting) and quantifying one or more microorganism from an intestinal content sample from the genus Lactobacillus. In this embodiment, a decreased population of one or more microorganism(s) of these genera in a sample obtained from the colon is an indicator of poor intestinal health, when compared to the level found in colon samples of non-challenged healthy control animals. However, an increased population of one or more microorganism(s) of these genera in a sample obtained from the caecum is an indicator of poor intestinal health, when compared to the level found in caecum samples of non-challenged healthy control animals.

Intestinal health can be determined in accordance with any number of means known in the art including, without limitation, measuring villus length; measuring villus-to crypt ratio; measuring T-lymphocyte infiltration in villi; and/or scoring the macroscopic gut appearance of the birds. Methods for determining intestinal health are described in detail in the Examples section. Similarly, quantification and identification of microorganisms can be conducted using any means known in the art, such as, but not limited to antibody based assays (for example, ELISA or Western Blot) or a PCR-based assay (for example, sequencing of the microbial 16S ribosomal DNA (rDNA) gene).

The invention can be further understood by reference to the following examples, which are provided by way of illustration and are not meant to be limiting.

### EXAMPLES

### Example 1: Assays

In the following examples, various assays were used as set forth below for ease in reading. Any deviations from the protocols provided below are indicated in the relevant sections. In these experiments, a spectrophotometer was used to measure the absorbance of the products formed after the completion of the reactions.

Histology: The duodenal loop was fixated in 4% formaldehyde for 24 hours, dehydrated in xylene and embedded in paraffin. Sections of 4 μm were cut using a microtome (Microme HM360, Thermo Scientific) and were processed as described by De Maesschalck et al. (2015). Villus length and crypt depth in the duodenum were determined by random measurement of twelve villi per intestinal segment using standard light microscopy (Leica DM LB2 Digita) and a computer based image analysis program, LAS V4.1 (Leica Application Suite V4, Germany). Afterwards the villus-to-crypt ratio was calculated. Antigen retrieval was performed on 4μm duodenal sections with a pressure cooker in citrate buffer (10 mM, pH 6). Slides were rinsed with washing buffer (Dako kit, K4011) and blocked with peroxidase reagent (Dako, 52023) for 5 minutes. Slides were rinsed with Aquadest and Dako washing buffer before incubation with anti-CD3 primary antibodies (Dako CD3, A0452) for 30 minutes at room temperature diluted 1:100 in antibody diluent (Dako, S3022). After rinsing again with washing buffer, slides were incubated with labelled polymer-HRP anti-rabbit (Envision+System-HRP, K4011) for 30 minutes at room temperature. Before adding di-amino-benzidine (DAB+) substrate and DAB+chromogen (Dako kit, K4011) for 5 minutes, slides were rinsed 2 times with washing buffer. To stop the staining, the slides were rinsed with Aquadest, dehydrated using the Shandon Varistain-Gemini Automated Slide Stainer and counterstained with hematoxylin for 10 seconds. The slides were analyzed with Leica DM LB2 Digital and a computer based image analysis program LAS V4.1 (Leica Application Suite V4, Germany) to measure CD3 positive area on a total area of 3 mm² which represents T-lymphocyte infiltration in approximately 10 villi per section.

Metabolomics: After freeze-drying of the colon and caecum content, 100 mg was weighted and resuspended in 2 ml ice cold 80% methanol. L-alanine d3 was used as internal standard. Herefore 25 μl of 100 ng/μl stock was added. Following vortexing (1 min) and centrifugation (10 min 9000 rpm) the supernatant was filtersterilized (0.45 μm) and diluted (1:3) with ultra-pure water. After vortexing (15 s) the filtrate was transferred into LC-MS vials.

An ultrahigh performance liquid chromatography hyphenated to Orbitrap HRMS (UHPLC-HRMS) was used for the chromatographic separation of the gastrointestinal (GIT)-derived metabolites using a Hypersil Gold column (1.9 μm, 100'2.1 mm) (Thermo Fisher Scientific, San-Francisco, USA) kept at 45° C. As binary solvent system, ultrapure water (A) and acetonitrile (B) both acidified with 0.1% formic acid were used and pumped at a flow rate of 400 μl min−1. The linear gradient program with the following proportions (v/v) of solvent A was applied: 0-1.5 min at 98%, 1.5-7.0 min from 98% to 75%, 7.0-8.0 min from 75% to 40%, 8.0-12.0 min from 40% to 5%, 12.0-14.0 min at 5%, 14.0-14.1 min from 5% to 98%, followed by 4.0 min of reequilibration. The injection volume of each sample was 10 μl.

HRMS analysis was performed on an Exactive stand-alone benchtop Orbitrap mass spectrometer (Thermo Fisher Scientific, San José, Calif., USA), equipped with a heated electrospray ionization source (HESI), operating in polarity switching mode. Ionization source working parameters were optimized and were set to a sheath, auxiliary, and sweep gas of 50, 25, and 5 arbitrary units (au), respectively, heater and capillary temperature of 350 and 250 ° C., and tube lens, skimmer, capillary, and spray voltage of 60 V, 20 V, 90 V, and 5 kV (±), respectively. A scan range of m/z 50-800 was chosen, and the resolution was set at 100 000 fwhm at 1 Hz. The automatic gain control (AGC) target was set at balanced (1×106 ions) with a maximum injection time of 50 ms.

Before and after analysis of samples, a standard mixture of 291 target analytes, with a concentration of 5 ng mL was injected to check the operational conditions of the device. To adjust for instrumental fluctuations, quality control (QC) samples (a pool of samples made from the biological test samples to be studied) were included. They were implemented at the beginning of the analytical run to stabilize the system and at the end of the sequence run for signal corrections within analytical batches. Targeted data processing was carried out with Xcalibur 3.0 software (Thermo Fisher Scientific, San Jose, Calif., USA), whereby compounds were identified based on their m/z-value, C-isotope profile, and retention time relative to that of the internal standard.

For untargeted data interpretation, the software package Sieve™ 2.2 (Thermo Fisher Scientific, San Jose, Calif., USA) was used to achieve automated peak extraction, peak alignment, deconvolution, and noise removal. This differential analysis was performed separately for the negative and positive ionization mode. As major parameters, a minimum peak intensity of 500 000 a.u., retention time width of 0.3 min, and mass window of 6 ppm were employed for feature extraction, with retention time, m/z-value and signal intensity as main feature descriptors. Normalization of the data set using the QC samples was performed to take instrumental drift into account.

Outputs of the targeted and untargeted data preprocessing were subjected to multivariate statistical, which was realized using Simca™ 14.1 software (Umetrics AB, Umea, Sweden). Principal component analysis (PCA) was performed for data exploration, to display the differentiation between the obtained fingerprints and potential outliers. This was followed by OPLS-DA to establish predictive models, which were validated by evaluating some quality parameters (R² (X) and Q² (Y), permutation testing (n ¼ 100), and cross-validated ANOVA (CV-ANOVA) (p-value<0.05).

DNA Extraction: DNA was extracted from caecum and colon content using the hexadecyltrimethylammonium bromide (CTAB) method as described previously (28, 29). To 100 mg of intestinal content, 0.5 g unwashed glass beads (Sigma-Aldrich, St. Louis, Mo.), 0.5 ml CTAB buffer (5% [wt/vol] hexadecyltrimethylammonium bromide, 0.35 M NaCl, 120 mM K2HPO4) and 0.5 ml phenol-chloroform-isoamyl alcohol mixture (25:24:1) (Sigma-Aldrich, St. Louis, Mo.) were added, followed by homogenization in a 2-ml destruction tube. The samples were shaken 6 times for 30 s each using a beadbeater (MagnaLyser; Roche, Basel, Switzerland) at 6,000 rpm with 30 s between shakings. After centrifugation (10 min, 8000 rpm), 300 μl of the supernatant was transferred to a new tube. The rest of the tube content was reextracted with 250 μl CTAB buffer and again homogenized with a beadbeater. The samples were centrifuged for 10 min at 8,000 rpm, and 300 μl supernatant was added to the first 300 μl supernatant. The phenol was removed by adding an equal volume of chloroform-isoamyl alcohol (24:1) (Sigma-Aldrich, St. Louis, Mo.) and performing a short spin. The aqueous phase was transferred to a new tube. The nucleic acids were precipitated with two volumes of polyethylene glycol (PEG) 6000 solution (30% [wt/vol] PEB, 1.6 M NaCl) for 2 h at room temperature. After centrifugation (20 min, 13,000 rpm), the pellet was rinsed with 1 ml of ice-cold 70% (vol/vol) ethanol. The pellet was dried and resuspended in 100 μl RNA-free water (VWR, Leuven, Belgium). The quality and the concentration of the DNA was examined spectrophotometrically (NanoDrop, Thermo Scientific, Waltham, Mass., USA).

Library Prep: DNA was extracted from caecum and colon content using the hexadecyltrimethylammonium bromide (CTAB) method as described previously (28, 29). To 100 mg of intestinal content, 0.5 g unwashed glass beads (Sigma-Aldrich, St. Louis, Mo.), 0.5 ml CTAB buffer (5% [wt/vol] hexadecyltrimethylammonium bromide, 0.35 M NaCl, 120 mM K2HPO4) and 0.5 ml phenol-chloroform-isoamyl alcohol mixture (25:24:1) (Sigma-Aldrich, St. Louis, Mo.) were added, followed by homogenization in a 2-ml destruction tube. The samples were shaken 6 times for 30 s each using a beadbeater (MagnaLyser; Roche, Basel, Switzerland) at 6,000 rpm with 30 s between shakings. After centrifugation (10 min, 8000 rpm), 300 μl of the supernatant was transferred to a new tube. The rest of the tube content was reextracted with 250 μl CTAB buffer and again homogenized with a beadbeater. The samples were centrifuged for 10 min at 8,000 rpm, and 300 μl supernatant was added to the first 300 μl supernatant. The phenol was removed by adding an equal volume of chloroform-isoamyl alcohol (24:1) (Sigma-Aldrich, St. Louis, Mo.) and performing a short spin. The aqueous phase was transferred to a new tube. The nucleic acids were precipitated with two volumes of polyethylene glycol (PEG) 6000 solution (30% [wt/vol] PEB, 1.6 M NaCl) for 2 h at room temperature. After centrifugation (20 min, 13,000 rpm), the pellet was rinsed with 1 ml of ice-cold 70% (vol/vol) ethanol. The pellet was dried and resuspended in 100 μl RNA-free water (VWR, Leuven, Belgium). The quality and the concentration of the DNA was examined spectrophotometrically (NanoDrop, Thermo Scientific, Waltham, Mass., USA).

To identify the taxonomic groups in the ileal, caecal and colon microbiota of the chickens, the V3-V4 hypervariable region of 16s rRNA gene was amplified using the gene-specific primers S-D-Bact-0341-b-S-17 (5′-TCGTCGGCAGCGTCAGATGTGTATAAGAGACAGCCTACGGGNGGCWGCAG-3′) and S-D-Bact-0785-a-A-21 (5′-GTCTCGTGGGCTCGGAGATGTGTATAAGAGACAGGACTACHVGGGTATCTAATCC-3′) (Klindworth, et al., 2013). Each 25 μl PCR reaction contained 2.5 μl DNA (˜5 ng/μl), 0.2 μM of each of the primers and 12.5 μl 2×KAPA HiFi HotStart ReadyMix (Kapa Biosystems, Wilmington, Mass., USA). The PCR amplification consisted of initial denaturation at 95° C. for 3 min, followed by 25 cycles of 95° C. for 30 s, 55° C. for 30 s, 72° C. for 30 s and a final extension at 72° C. for 5 min. The PCR products were purified using CleanNGS beads (CleanNA, Waddinxveen, The Netherlands). The DNA quantity and quality was analyzed spectrophotometrically (NanoDrop) and by agarose gel electrophoresis. A second PCR step was used to attach dual indices and Illumina sequencing adapters in a 50 μl reaction volume containing 5 μl of purified PCR product, 2× KAPA HiFi HotStart ReadyMix (25 μl) and 0.5 μM primers. The PCR conditions were the same as the first PCR with the number of cycles reduced to 8. The final PCR products were purified and the concentration was determined using the Quantus double-stranded DNA assay (Promega, Madison, Wis., USA). The final barcoded libraries were combined to an equimolar 5 nM pool and sequenced with 30% PhiX spike-in using the Illumina MiSeq v3 technology (2×300 bp, paired-end) at the Oklahoma Medical Research Center (Oklahoma City, Okla., USA) for samples from trial 1 and at Macrogen (Seoul, Korea) for samples from trial 2.

### Bioinformatics and Statistical Analysis of 16S rRNA Gene Amplicon Data

Demultiplexing of the amplicon dataset and deletion of the barcodes was done by the sequencing provider. Quality of the raw sequence data was checked with the FastQC quality-control tool (Babraham Bioinformatics, Cambridge, United Kingdom; http://www.bioinformatics.babraham.ac.uk/projects/fastqc) followed by initial quality filtering using Trimmomatic v0.38 by cutting reads with an average quality per base below 15 using a 4-base sliding window and discarding reads with a minimum length of 200 bp (Bolger, et al., 2014). The paired-end sequences were assembled and primers were removed using PANDAseq (Masella, et al., 2012), with a quality threshold of 0.9 and length cut-off values for the merged sequences between 390 and 430 bp. Chimeric sequences were removed using UCHIME (Edgar, et al., 2011). Open-reference operational taxonomic unit (OTU) picking was performed at 97% sequence similarity using USEARCH (v6.1) and converted to an OTU table (Edgar, 2010). OTU taxonomy was assigned against the Silva database (v128, clustered at 97% identity) (Quast, et al., 2013) using the PyNast algorithm with QIIME (v1.9.1) default parameters (Caporaso, et al., 2010). OTUs with a total abundance below 0.01% of the total sequences were discarded (Bokulich, et al., 2013), resulting in an average of approximately 26920 reads per sample. Alpha rarefaction curves were generated using the QIIME “alpha_rarefaction.py” script and in trial 1 a subsampling depth of 15 000 reads was selected. One ileal sample from the control group was excluded from further analysis due to insufficient sequencing depth. Any sequences of mitochondrial or chloroplastic origins were removed. In trial 2 a subsampling depth of 9900 reads was selected. One caecal sample from the control group and one caecal sample from the challenge group was excluded from further analysis due to insufficient sequencing depth. Any sequences of mitochondrial or chloroplastic origins were removed.

Further analysis of alpha diversity (Observed OTUs, Chaol richness estimator and Shannon diversity estimator) and beta diversity (Bray-Curtis dissimilarities) were performed using the phyloseq (McMurdie and Holmes, 2013) pipeline in R (v3.4.3). Normality of the alpha diversity data was tested using the Shapiro-Wilk test. A t-test was used for normal distributed data, whereas the Mann-Whitney U test was used for not normal distributed data. Differences in beta diversity were examined using the anosim function from the vegan package. Differences in relative abundance at the phylum level were assessed using the two-sided Welch t-test from the mt wrapper in phyloseq, with the P-value adjusted for multiple hypothesis testing using the Benjamini-Hochberg method. To detect differentially abundant taxa between the control and challenge group, both DESeq2 analysis and Linear Discriminant Analysis (LDA) Effect Size (LEfSe) analysis were used. DESeq2 was applied on the non-rarified community composition data for either caecal or ileal communities (Love, et al., 2014). Significant differences were obtained using a Wald test followed by a Benjamini-Hochberg multiple hypothesis correction. LEfSe analysis was performed on Genus level using the LEfSe wrapper “koeken.py” with an ANOVA p-value<0.05 and logarithmic LDA score threshold of 2.0 (Segata et al., 2011). The correlation of bacterial taxa with different bird characteristics (body weight, dysbiosis score, coccidiosis score, or histological parameters (crypt depth, villus length, villus-to-crypt ratio or CD3 area percentage)) was assessed using the QIIME “observation_metadata_correlation.py” script. For each group (control or challenge) and each intestinal segment (ileum, caecum or colon), the Spearman correlation coefficient was calculated using the relative abundance of all families and genera versus each bird parameter. The resulting p-values were corrected by the Benjamini-Hochberg FDR procedure for multiple comparisons. For all tests, a P-value <0.05 was considered significant.

### Example 2: Induction of Dysbiosis in Chickens with Challenge Model Trials

A total of 360 day-old broilers (Ross 308) were obtained from a local hatchery and housed in floor pens on wooden shavings. Throughout the study, feed and drinking water were provided ad libitum. The broilers were randomly assigned to two treatment groups, a control and challenge group (9 pens per treatment and 20 broilers per pen). All animals were fed a commercial feed till day 12 and the feed was switched to a wheat (57.5%) based diet supplemented with 5% rye. From day 12 to 18, all animals from the challenge group received 10 mg florfenicol and 10 mg enrofloxacin per kg body weight via the drinking water daily, to induce substantial changes in the gut microbial community. After the antibiotic treatment, 1 ml of a bacterial cocktail consisting of 10⁹ cfu Escherichia coli (G.78.71), 10¹⁰ cfu Enterococcus sp. (G.78.62), 10⁹ cfu Lactobacillus salivarius (LMG22873), 10⁸ cfu Lactobacillus crispatus (LMG49479), 10⁸ cfu Clostridium perfringens (netB-) (D.39.61) and 10⁸ cfu Ruminococcus gnavus (LMG27713) was given daily by oral gavage from day 19 till 21. On day 20, the animals were administered a coccidial challenge consisting of different Eimeria sp., namely 60.000 oocysts of E. acervulina and 30.000 oocysts E. maxima. At day 26, the birds were weighed and 3 birds per pen were euthanized. The duodenal loop was sampled for histological examination and content from ileum and ceacum was collected for metabolomic analysis.

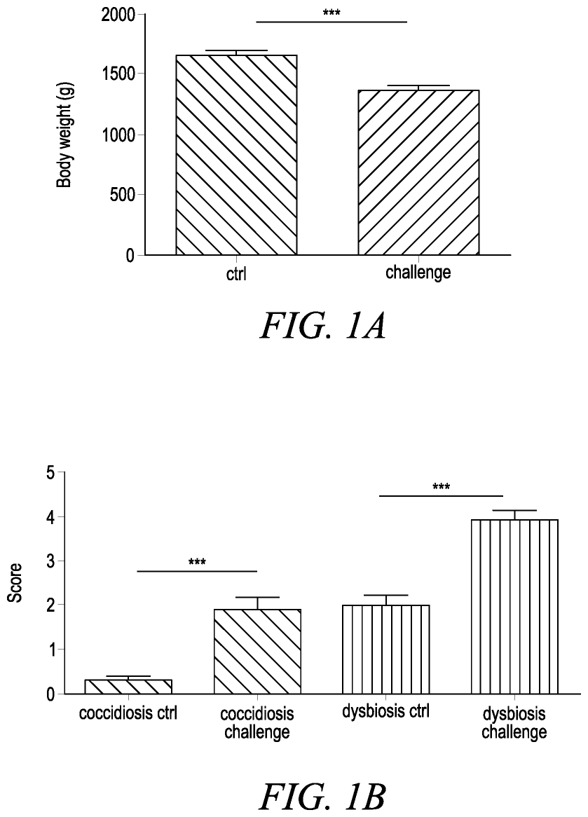

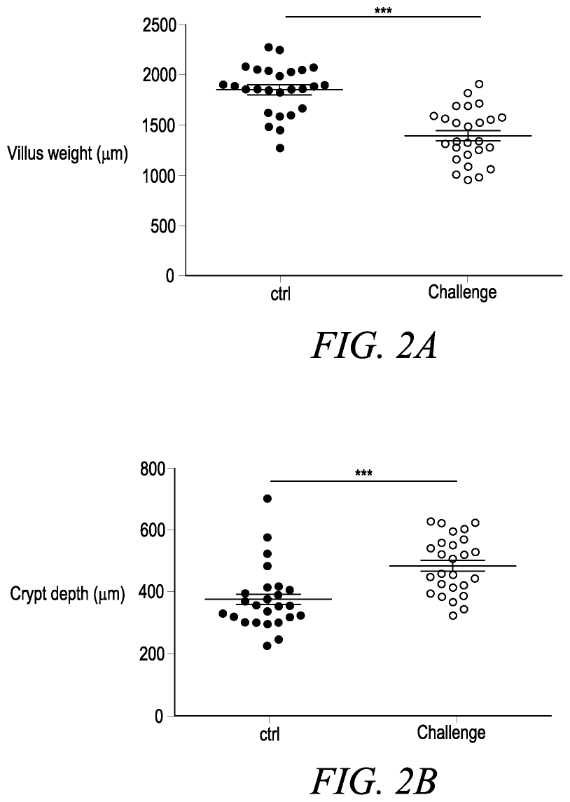

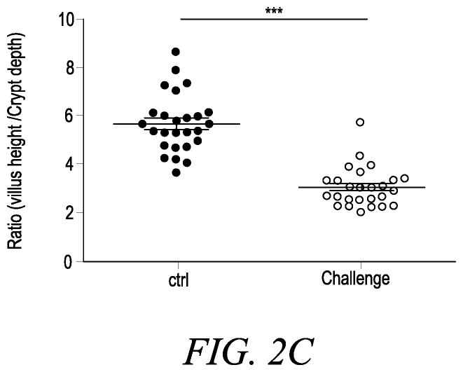

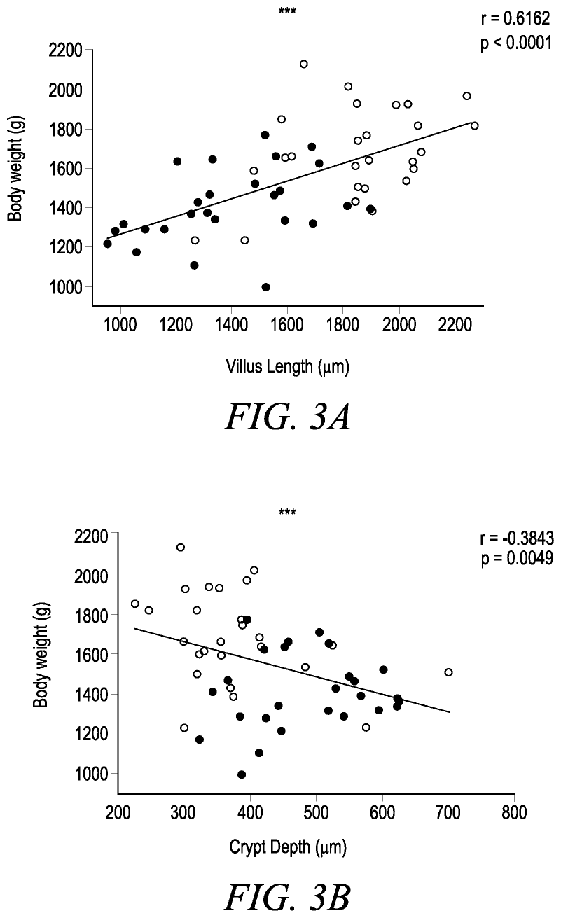

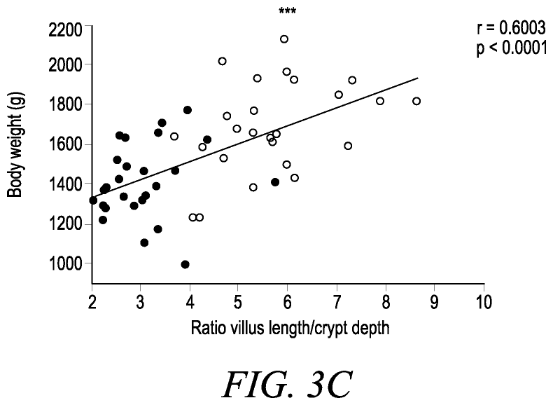

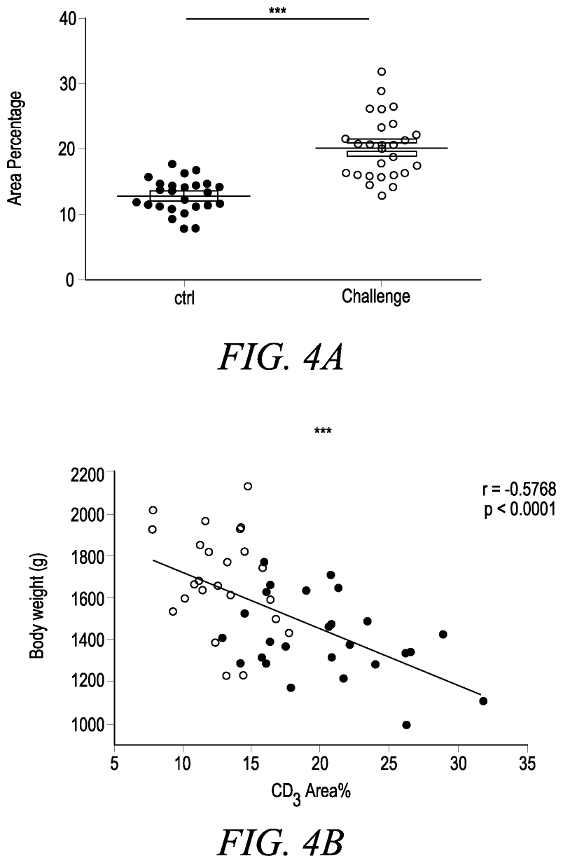

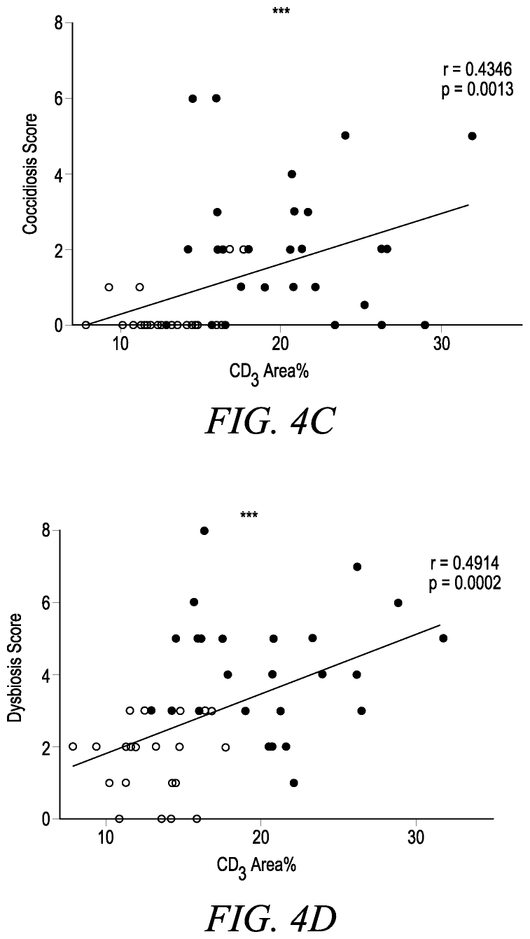

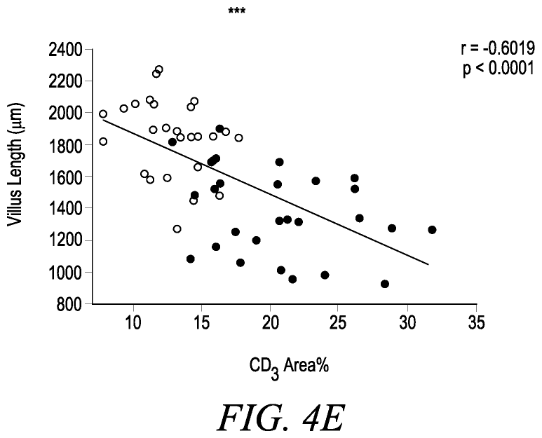

Challenged birds exhibited significant body weight reductions (FIG. 1A) as well as increased dysbiosis and coccidiosis score (FIG. 1B) each performed blindly according to De Gussem (2010; “Macroscopic scoring system for bacterialenteritis in broiler chickens and turkeys;” In WVPA Meeting (2010), Merelbeke, Belgium) and Johnson & Reid (1970; Exp. Parasitol. 28:30-36) the disclosures of which are incorporated herein, respectively. Histological evaluation revealed that challenged birds significantly decreased villus length (FIG. 2A) and increased crypt depth (FIG. 2B; see also FIG. 2C). In particular, decreased villus length and increased crypt depth were both associated with decreased bird body weight (FIG. 3A, FIG. 3B, and FIG. 3C). Moreover, challenged birds exhibited significantly increased intestinal immune cell infiltration relative to control animals (FIG. 4A) which was correlated with decreased body weight (FIG. 4B), increased coccidiosis and dysbiosis score (FIG. 4C and FIG. 4D), and villus length (FIG. 4E). Overall, these data suggest that challenged animals exhibited significantly decreased weight and other morphological and histological symptoms associated with intestinal dysbiosis and coccidiosis.

Next, a second trial was performed using a modified diet. A total of 676 day-old broilers (Ross 308) were obtained from a local hatchery and housed in floor pens on wooden shavings. Throughout the study, feed and drinking water were provided ad libitum. The broilers were randomly assigned to two treatment groups, a control and challenge group (13 pens per treatment and 26 broilers per pen). All animals were fed a commercial feed till day 14 and the feed was switched to a wheat based diet supplemented with 20% triticale. From day 14 to 20, all animals from the challenge group received 10 mg florfenicol and 10 mg enrofloxacin per kg body weight via the drinking water daily, to induce substantial changes in the gut microbial community. After the antibiotic treatment, 1 ml of a bacterial cocktail consisting of 10⁸ cfu Escherichia coli (G.78.71), 10⁸ cfu Enterococcus sp. (G.78.62), 10⁸ cfu Lactobacillus salivarius (LMG22873), 10⁷ cfu Lactobacillus crispatus (LMG49479), and 10⁸ cfu Clostridium perfringens (netB-) (D.39.61) was given daily by oral gavage from day 21 till 23. On day 22, the animals were administered a coccidial challenge consisting of 60.000 oocysts of E. acervulina and 30.000 oocysts E. maxima. At day 28, the birds were weighed and 3 birds per pen were euthanized. The duodenal loop was sampled for histological examination and content from caecum and colon was collected for DNA extraction and metabolomics.

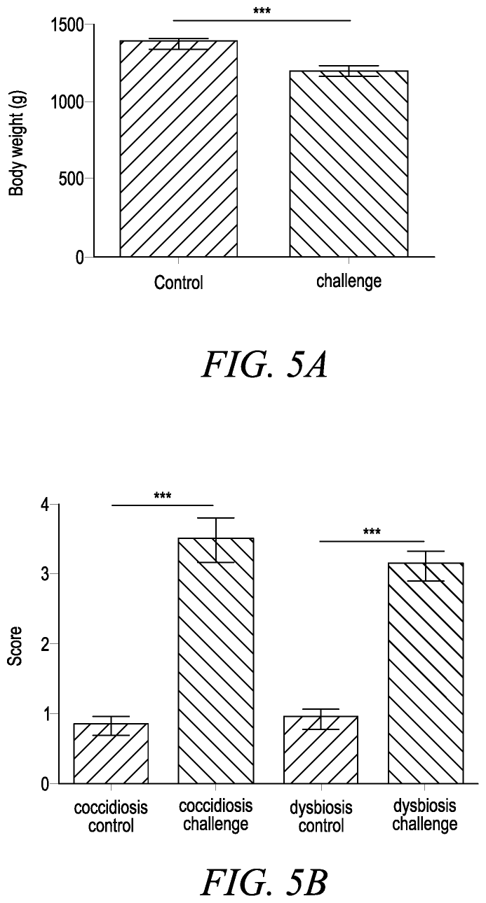

Challenged birds exhibited significant body weight reductions (FIG. 5A) as well as increased dysbiosis and coccidiosis score (FIG. 5B). Similar to the results displayed in FIG. 2 to FIG. 4 in the first trial, histological evaluation revealed that challenged birds had significantly decreased villus length and increased crypt depth. Decreased villus length and increased crypt depth were both associated with decreased bird body weight. Moreover, challenged birds exhibited significantly increased intestinal immune cell infiltration relative to control animals which was correlated with decreased body weight, increased coccidiosis and dysbiosis score, and villus length.

### Example 3: Identification of Metabolic Biomarkers Correlated with Intestinal Health

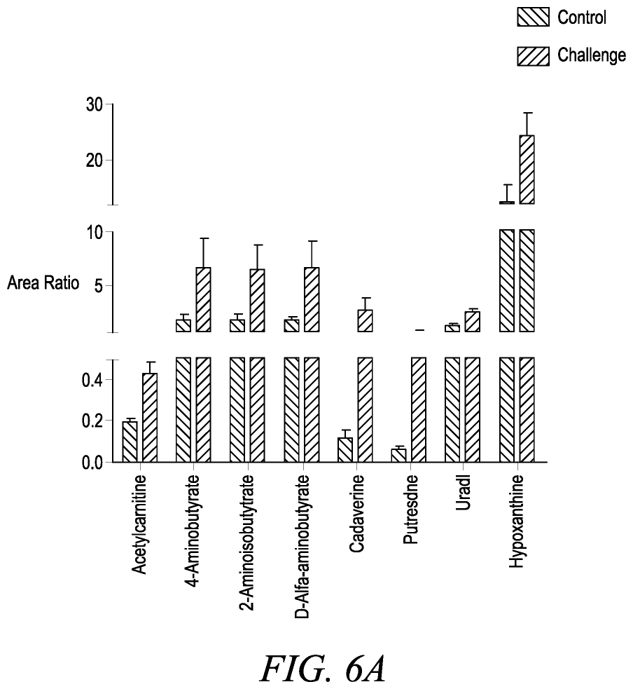

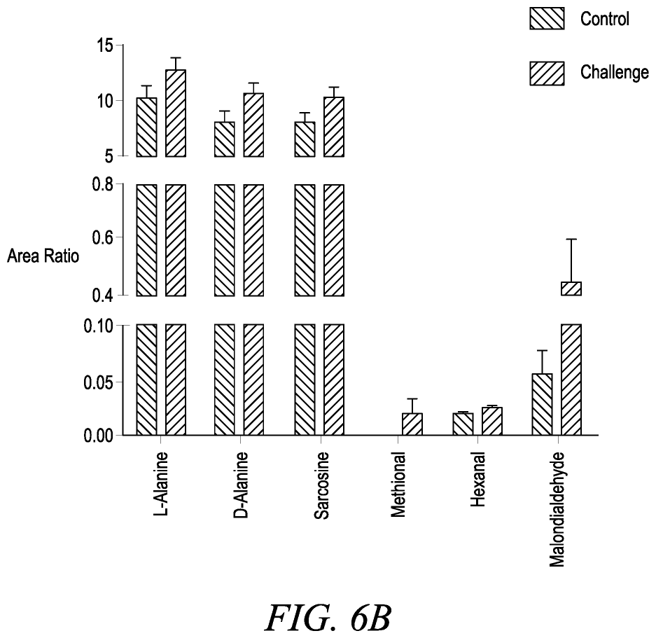

A metabolomic analysis of colon and caecum samples derived from the control and challenged animals of Example 2 was performed. As shown in FIG. 6A and FIG. 6B, a number of metabolites were observed in both the colon (FIG. 6A) and caecum (FIG. 6B) of challenged chickens at levels significantly higher in comparison to their corresponding levels in control chickens. In addition to the metabolites shown in FIG. 6A and FIG. 6B, the following additional compounds were found in the intestines of challenged chickens at levels significantly higher than those found in unchallenged controls: linoleyl carnitine, linalool, 3-[(9Z)-9-octadecenoyloxy]-4-(trimethylammonio)butanoate, (−)-trans-methyl dihydrojasmonate, icomucret, 1,3-dioctanoylglycerol, and ethyl 2-nonynoate. Thus, the presence of one or more of these compounds at levels significantly higher than healthy control animals is correlated with poor intestinal health and their presence and quantification can be used to assess and predict the intestinal health of poultry.

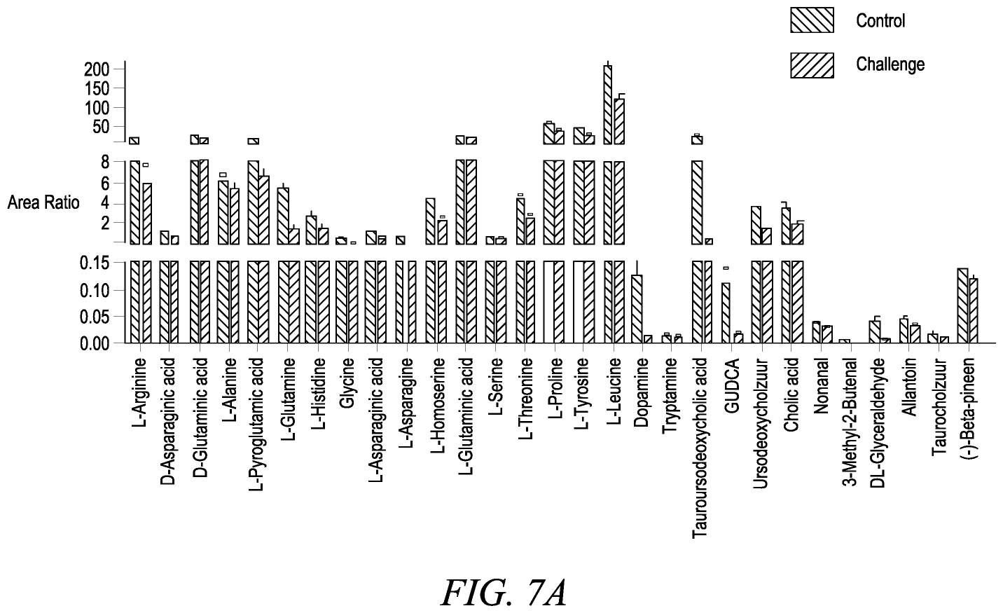

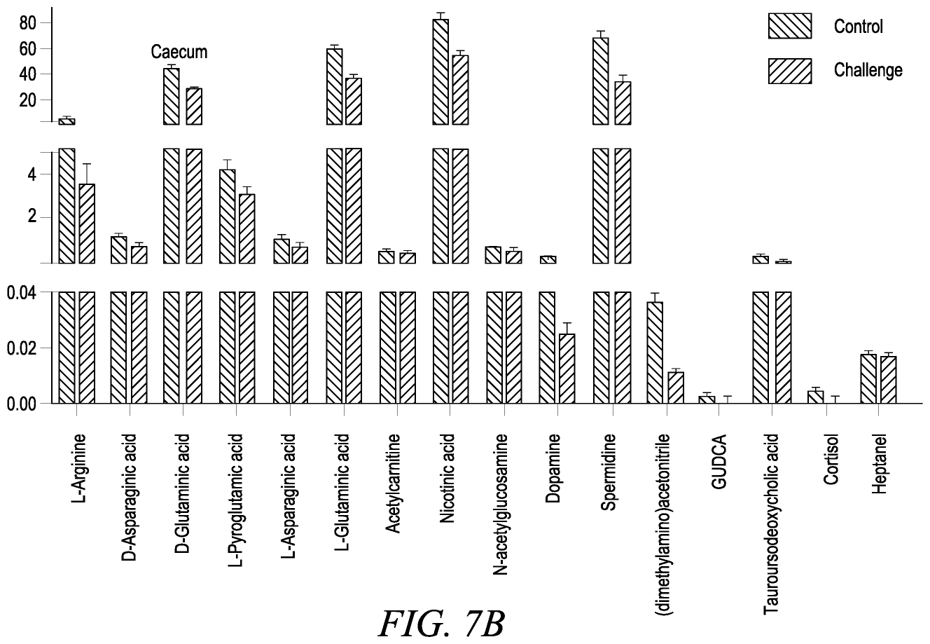

As shown in FIG. 7A and FIG. 7B, additional metabolites were identified in both the colon (FIG. 7A) and caecum (FIG. 7B) of challenged chickens at levels significantly lower in comparison to their corresponding levels in control chickens (i.e., these compounds were present at statistically significant higher levels in healthy unchallenged animals). In addition to the metabolites shown in FIG. 7A and FIG. 7B, the following additional compounds were found in the intestines of challenged chickens at levels significantly lower than those found in unchallenged controls (i.e., these compounds are more present in healthy unchallenged control animals): 5-(2-carboxyethyl)-2-hydroxyphenyl beta-D-glucopyranosiduronic acid, 4,15-Diacetoxy-3-hydroxy-12,13-epoxytrichothec-9-en-8-yl3-hydroxy-3-methylbutanoate, scoparone, asp-leu, ethyl benzoylacetate, L-(+)-glutamine, 1-allyl-2,3,4,5-tetramethoxybenzene, (DL)-3-O-methyldopa, dictyoquinazol A, 1-(3-furyl)-7-hydroxy-4,8-dimethyl-1,6-nonanedione, methyl 3,4,5-trimethoxycinnamate, and butylparaben. Thus, the presence of one or more of these compounds at levels significantly lower than healthy control animals is correlated with poor intestinal health and their presence and quantification can be used to assess and predict the intestinal health of poultry.

Certain metabolites were found to be differentially expressed in either the colon or the caecum between challenged and healthy control animals. Specifically, with respect to the colon, the following metabolites were found to be present in greater quantities in the colon of healthy control animal: 5-(2-Carboxyethyl)-2-hydroxyphenyl beta-D-glucopyranosiduronic acid, 4,15-Diacetoxy-3-hydroxy-12,13-epoxytrichothec-9-en-8-yl3-hydroxy-3-methylbutanoate, Scoparone, asp-leu, Ethyl benzoylacetate, L-(+)-glutamine. In contrast, the following metabolites were found to be present in greater quantities in the colon of challenged animals: Linoleyl carnitine, Linalool, 3-[(9Z)-9-Octadecenoyloxy]-4-(trimethylammonio)butanoate, (−)-trans-Methyl dihydrojasmonate, icomucret, 1,3-Dioctanoylglycerol.

A further non-limiting list of differentially quantified metabolites between challenged and unchallenged birds in the colon can be found in Table 2.

TABLE 2
Differentially quantified colon metabolites

| Significantly more in | | | |
|---|---|---|---|
| challenged chickens | Significantly more in unchallenged chickens | | |
| Acetylcarnitine | L-Arginine | L-Asparagine | Typtamine |
| 4-Aminobutyrate | Aspartic acid | L-Homoserine | Tauroursodeoxychdic acid |
| 2-Amino-isobutyrate | Glutamic acid | L-Serine | Glycoursodeoxycholic acid |
| D-Alfa-aminobutyrate | L-Alanine | L-Threonine | Ursodeoxycholic acid |
| Cadaverine | L-Pyroglutamic acid | L-Proline | Cholic acid |
| Putrescine | L-Glutamine | L-Tyrosine | Nonanal |
| Uracil | L-histidine | L-Leucine | 3-Methyl-2-butenal |
| Hypoxanthine | (−)-beta-pineen | Taurocholic acid | Allantoin |

Regarding the caecum, the following metabolites were found to be present in greater quantities in the caecum of healthy control animal: 1-Allyl-2,3,4,5-tetramethoxybenzene, (DL)-3-O-Methyldopa, dictyoquinazol A, 1-(3-Furyl)-7-hydroxy-4,8-dimethyl-1,6-nonanedione, Methyl 3,4,5-trimethoxycinnamate, and Butylparaben. In contrast Ethyl 2-nonynoate was found to be present in greater quantities in the caecum of challenged animals.

A further non-limiting list of differentially quantified metabolites between challenged and unchallenged birds in the colon can be found in Table 3.

TABLE 3
Differentially quantified colon metabolites

| | |
|---|---|
| Significantly more in | |
| challenged chickens | Significantly more in unchallenged chickens |

| | | |
|---|---|---|
| L-Alanine | L-Arginine | Dopamine |
| D-Alanine | Aspartic acid | Spermidine |
| Sarcosine | Glutamic acid | (Dimethlyamino)acetonitrile |
| Methional | L-Pyroglutamic acid | Glycoursodeoxycholic acid |
| Hexanal | Acetylcarnitine | Tauroursodeoxycholic acid |
| Malondiadehyde | N-acetylglucosamine | Heptanal |

### Example 4: Identification of Microbial Biomarkers for Intestinal Health

With respect to the initial trial described in Example 2, statistical analysis of 16S rRNA gene amplicon data was used to identify the taxonomic groups of bacteria in the ileal and caecal microbiota of control and challenged chickens as well as statistically significant changes in their populations following challenge. The results are shown in Table 4.

TABLE 4
Microbiome changes in challenged birds in ileum and caecum

| | | Ileum | | | Caecum | | |
|---|---|---|---|---|---|---|---|
| Taxa | Bacteria | Control | Challenge | p value | Control | Challenge | p value |
| Family | Clostridiales vadinBB60 | | | | 7.04 | 2.54 | 0.001 |
| | group | | | | | | |
| Family | Peptostreptococcaceae | 0.52 | 0.00 | 0.0024 | 0.02 | 0.00 | 0.000 |
| Genus | Brevibacterium | 0.13 | 0.01 | 0.0024 | | | |
| Genus | Ambiguous_taxa | 0.52 | 0.00 | 0.0024 | | | |
| | (Peptostreptococcaceae) | | | | | | |
| Genus | Brachybacterium | 0.10 | 0.01 | 0.0016 | | | |
| Genus | Ruminiclostridium 5 | | | | 1.37 | 0.79 | 0.001 |
| Genus | Candidatus Arthromitus | 1.14 | 0.41 | 0.0023 | | | |
| Genus | [Ruminococcus] torques | | | | 2.27 | 1.72 | 0.063 |
| | group | | | | | | |
| Genus | Ruminiclostridium | 0.06 | 0.02 | 0.0468 | 0.64 | 0.08 | <0.0001 |
| Genus | uncultured bacterium | | | | 6.97 | 2.49 | 0.001 |
| | (Clostridiales vadinBB60 | | | | | | |
| | group) | | | | | | |
| Genus | Ruminococcus 1 | | | | 0.32 | 0.13 | 0.004 |
| Genus | Defluviitaleaceae UCG-011 | | | | 0.13 | 0.03 | 0.001 |
| Genus | Streptococcus | | | | 0.06 | 0.00 | 0.038 |
| Genus | Shuttleworthia | | | | 0.35 | 0.13 | 0.001 |
| Genus | Lachnoclostridium | | | | 1.22 | 0.31 | <0.0001 |
| Genus | Lactobacillus | | | | 13.19 | 20.88 | 0.051 |
| Genus | Lachnospiraceae NK4A136 | | | | 0.53 | 0.07 | <0.0001 |
| | group | | | | | | |
| Genus | Ruminococcaceae UCG-005 | | | | 0.76 | 0.31 | 0.001 |

Regarding the second trial using a modified diet, statistical analysis of 16S rRNA gene amplicon data was used to identify the taxonomic groups of bacteria in the colonic and caecal microbiota of control and challenged chickens as well as statistically significant changes in their populations following challenge. The results are shown in Table 5.

TABLE 5
Microbiome changes in challenged birds in colon and caecum

| | | Colon | | | Caecum | | |
|---|---|---|---|---|---|---|---|
| Taxa | Bacteria | Control | Challenge | p value | Control | Challenge | p value |
| Family | Clostridiales vadinBB60 | | | | 0.11% | 0.08% | 0.0001 |
| | group | | | | | | |
| Family | Peptostreptococcaceae | 0.30% | 0.00% | 0.0001 | | | |
| Genus | Brevibacterium | 0.28% | 0.05% | 0.0594 | | | |
| Genus | Ambiguous_taxa | 0.30% | 0.00% | 0.0001 | | | |
| | (Peptostreptococcaceae) | | | | | | |
| Genus | Brachybacterium | 0.57% | 0.02% | 0.0015 | | | |
| Genus | Ruminiclostridium 5 | 1.01% | 0.50% | 0.0686 | 1.87% | 0.91% | 0.006 |
| Genus | Candidatus Arthromitus | 1.13% | 0.00% | P < 0.0001 | | | |
| Genus | [Ruminococcus] torques | 1.55% | 3.53% | 0.0059 | | | |
| | group | | | | | | |
| Genus | uncultured bacterium | | | | 0.11% | 0.08% | 0.0001 |
| | (Clostridiales vadinBB60 | | | | | | |
| | group) | | | | | | |
| Genus | Ruminococcus 1 | 0.07% | 0.03% | 0.1294 | | | |
| Genus | Defluviitaleaceae UCG-011 | 0.10% | 0.12% | 0.0293 | 0.23% | 0.09% | 0.0056 |
| Genus | Streptococcus | 0.21% | 0.02% | 0.0012 | | | |
| Genus | Shuttleworthia | 0.20% | 0.17% | 0.0089 | 0.34% | 0.09% | 0.0004 |
| Genus | Lachnoclostridium | 0.77% | 1.26% | 0.0262 | | | |
| Genus | Lactobacillus | 45.14% | 32.71% | 0.0774 | 3.84% | 7.41% | 0.0653 |
| Genus | Lachnospiraceae NK4A136 | | | | 0.49% | 0.20% | 0.0083 |
| | group | | | | | | |
| Genus | Ruminococcaceae UCG-005 | | | | 3.67% | 1.42% | 0.0017 |

1-18. (canceled)

19. A method for detecting and/or quantifying one or more metabolite(s) from a domesticated bird at risk for or thought to be at risk for poor intestinal health comprising:
detecting and/or quantifying one or more metabolites in a sample selected from the group consisting of linoleyl carnitine, linalool, 3-[(9Z)-9-octadecenoyloxy]-4-(trimethylammonio)butanoate, (−)-trans-methyl dihydrojasmonate, icomucret, 1,3-dioctanoylglycerol, ethyl 2-nonynoate, L-arginine, 4-aminobutyrate, 2-amino-isobutyrate, D-alpha-aminobutyrate, cadaverine, putrescine, uracil, hypoxanthine, D-alanine, sarcosine, methional, hexanal, malondialdehyde, L-alanine, and acetylcarnitine, wherein the sample is a fecal or an intestinal content sample.

20. The method of claim 19, further comprising detecting and/or quantifying one or more metabolites in the sample selected from the group consisting of 5-(2-carboxyethyl)-2-hydroxyphenyl beta-D-glucopyranosiduronic acid, 4,15-Diacetoxy-3-hydroxy-12,13-epoxytrichothec-9-en-8-yl 3-hydroxy-3-methylbutanoate, scoparone, asp-leu, ethyl benzoylacetate, L-(+)-glutamine, 1-allyl-2,3,4,5-tetramethoxybenzene, (DL)-3-0-methyldopa, dictyoquinazol A, 1-(3-furyl)-7-hydroxy-4,8-dimethyl-1,6-nonanedione, methyl 3,4,5-trimethoxycinnamate, butylparaben, aspartic acid, glutamic acid, L-pyroglutamic acid, L-glutamine, L-histidine, glycine, (−)-beta-pineen, L-asparagine, L-homoserine, L-serine, L-threonine, L-proline, L-tyrosine, L-leucine, dopamine, taurocholic acid, typtamine, tauroursodeoxychdic acid, glycoursodeoxycholic acid, ursodeoxycholic acid, cholic acid, nonanal, 3-methyl-2-butenal, DL-glyceraldehyde, allantoin, nicotinic acid, N-acetylglucosamine, spermidine, (dimethlyamino)acetonitrile, glycoursodeoxycholic acid, tauroursodeoxycholic acid, cortisol, and heptanal.

21. The method of claim 19, further comprising detecting and/or quantifying populations of one or more microorganism(s) in a fecal and/or intestinal content sample from the bird selected from the group consisting of: a microorganism from the Clostridiales vadinBB60 group family of microorganisms and a microorganism from the Peptostreptococcaceae family of microorganisms.

22. The method of claim 19, further comprising detecting and/or quantifying populations of one or more microorganism(s) in a fecal and/or intestinal content sample from the bird selected from the group consisting of: a microorganism from the genus Brevibacterium, Brachybacterium, Ruminiclostridium, Candidatus Arthromitus, Ruminococcus with the exception of Ruminococcus torques, Streptococcus, Shuttleworthia, Lachnospiraceae NK4A136 group, and Ruminococcaceae UCG-005.

23. The method of claim 19, further comprising detecting and/or quantifying populations of one or more microorganism(s) in an intestinal content sample from the bird selected from: a microorganism from the genus Defluviitaleaceae UCG-011, a microorganism from the genus Lachnoclostridium, or a microorganism from the Ruminococcus torques group.

24. The method of claim 19, further comprising detecting and/or quantifying populations of one or more microorganism(s) in an intestinal content sample from the bird a microorganism from the genus Lactobacillus.

25. The method of claim 19, wherein the intestinal content sample is obtained from ileum, colon, or caecum.

26. The method of claim 19, further comprising (a) measuring villus length in the duodenum of the birds; (b) measuring villus-to crypt ratio in the duodenum of the birds; (c) measuring T-lymphocyte infiltration in villi;
and/or (d) scoring the macroscopic gut appearance of the birds.

27. The method of claim 19, wherein the domesticated bird is selected from the group consisting of chickens, turkeys, ducks, geese, quail, emus, ostriches, and pheasant.

28. The method of claim 27, wherein the chicken is a broiler.

29. The method of claim 19, wherein said one or more metabolite(s) and/or said populations of one or more microorganism(s) are quantified by using antibodies which specifically bind to said metabolite.

30. The method of claim 29, wherein said antibodies are part of an Enzyme-Linked Immuno Sorbent Assay (ELISA).

31. The method of claim 19, wherein said one or more metabolite(s) are quantified by using gas chromatography-mass spectrometry (GC-MS), nuclear magnetic resonance (NMR), liquid chromatography-mass spectrometry (LC-MS) or HPLC.

32. The method of claim 21, wherein said one or more microorganisms are identified and quantified by real-time PCR.

33. The method of claim 32, further comprising sequencing the 16S ribosomal DNA (rDNA) gene.
